(19) United States
(12) Patent Application Publication
(10) Pub. No.: US 2025/0249151 A1
(43) Pub. Date: Aug. 7, 2025

(54) DEVICES AND METHODS FOR BONE FRACTURE AND SEGMENTAL DEFECT HEALING

(71) Applicant: The Trustees of the University of Pennsylvania, Philadelphia, PA (US)

(72) Inventors: Annemarie Lang, Ann Arbor, MI (US); Joel D. Boerckel, Bala Cynwyd, PA (US)

(21) Appl. No.: 19/040,493

(22) Filed: Jan. 29, 2025

Related U.S. Application Data
(60) Provisional application No. 63/626,981, filed on Jan. 30, 2024.

(51) Int. Cl. A61L 27/54; A61L 27/26; A61L 27/58

(52) U.S. Cl. CPC A61L 27/54; A61L 27/26; A61L 27/58; A61L 2300/256; A61L 2430/02

(57) ABSTRACT

Provided herein are devices and methods that represent a dual function approach to tissue engineering and regenerative medicine, particularly for bone fracture or segmental defect healing. The devices include an implantable substrate; a first active composition, and a second active composition that comprises one or more oxygen-modulating compositions. The incorporation of dual bioactive compositions on the substrate offers a versatile platform for manipulating cells and modulating the local tissue oxygen environment at the site of implantation.

### CROSS REFERENCE TO RELATED APPLICATIONS

This application claims priority to and the benefit of U.S. Provisional Patent Application No. 63/626,981, filed Jan. 30, 2024, the disclosure of which is incorporated herein by reference in its entirety for any and all purposes.

### GOVERNMENT RIGHTS

This invention was made with government support under AR074948 awarded by the National Institutes of Health. The government has certain rights in the invention.

### TECHNICAL FIELD

This invention is generally related to the field of bone fracture healing with devices and methods that modulate erythropoiesis and oxygen levels at the fracture site.

### BACKGROUND

Bone regeneration is a challenging clinical problem. Each year, millions of patients worldwide experience bone fractures one every two-to-three seconds. Over 10-15% of these fractures suffer from impaired healing, resulting in estimated costs of over $10,000 per treatment (1) and the average disablement duration for limb fractures is estimated with 50 days (2). Segmental defects, in which a segment of bone must be removed due to trauma or surgical resection, are particularly challenging, requiring treatment by bone grafts or other means. Variations of segmental defects are corrective surgical procedures such as osteotomies (e.g. to treat hip dysplasia) and distraction osteogenesis, which is frequently used in the craniofacial area. More than 1.5 million bone grafts are performed annually in the United States, making bone the second-most grafted tissue behind blood transfusion (3). However, insufficient neovascularization, the formation of new blood vessels, and efficient recruitment of osteoblasts to the injury site remain the most pressing challenges for clinical bone repair and regeneration. (4-6) The osteoprogenitor cells that mediate bone repair mobilize via vascular invasion (7, 8). This is a major bottleneck for clinical outcomes.

Currently, 10% of patients with fractures suffer from delayed healing or non-union, leading to immobility, pain and a loss in quality of life- and to a significant economic burden for the society (9, 10). Patients with fracture healing disorders often require several further revision surgeries. Normal fracture healing should complete within 4 months. If healing takes longer, it is termed ‘delayed’. If bridging of the fracture gap does not take place after 9 months, it is termed ‘non-union’ (11). The inability to predict which fractures will go on to delayed- or non-union, and to induce consistent fracture healing outcomes, is a major clinical problem.

Primary current treatment strategies feature autologous and allogeneic bone grafts. Autologous bone grafts are bone fragments taken from a different site, from the same patient. The most common donor site is the iliac crest (the top of the pelvis). However, the amount of bone graft material that can be removed is limited, and donor site pain and morbidity are significant. Therefore, allogeneic bone grafts (devitalized bone taken from a cadaver donor) are often used, though these frequently fail to revitalize leading to re-fracture. The only current clinically-used bioengineered solution for this problem is recombinant human (rh) bone morphogenetic protein-2 (BMP-2) for local delivery into the fracture gap (12). This is a product of Medtronic, Inc., marketed under the name “Infuse™.” Infuse™ consists of a scaffold matrix composed of lyophilized collagen and a recombinant protein solution, making it an easy-to-use off-the-shelf biological device. However, rhBMP-2 has significant side effects, including stimulation of inflammation, significant risks of heterotopic ossification (bone formation outside of the desired site in the skeleton, for example in the surrounding muscle), and tumorigenesis (13, 14). Thus, a similar product based on rhBMP-7 has been withdrawn from the market, and the use of rhBMP-2 is restricted and the FDA recommends no off-label use (15).

Therefore, there remains a need for compositions and methods for bone regenerative therapy that could overcome these challenges for fracture healing and large (segmental) bone defect regeneration.

### SUMMARY

Provided herein are devices and methods for healing bone fractures at bone fracture or segmental defect sites. The devices can comprise orthopedic devices that comprise an implantable substrate; a first active composition, the first active composition optionally in contact with the implantable substrate; and a second active composition, the second active composition optionally in contact with the implantable substrate. The first active composition can comprise one or more compositions for bone union. The second active composition can comprise one or more oxygen-modulating compositions.

Also provided are methods of enhancing healing at a bone fracture or segmental defect site in subjects. The methods can comprise administering at the bone fracture or segmental defect site any one or more of the devices provided herein.

Also disclosed are methods of reducing incidences of non-union among subjects with bone fractures or segmental defects. The methods can comprise administering at the bone fracture or segmental defect site of the subjects any one or more of the devices provided herein. The provided devices can reduce the incidence of non-union at the bone fracture or segmental defect site among the subjects.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1E are graphs depicting staining of intracellular oxygenation in murine bone marrow using EF5 staining. FIGS. 1A and 1B—Schematic overview, representative histograms, and quantified cell frequencies in vitro. (N=8). One-Way ANOVA with pre-selected pairs; p **<0.01. FIG. 1C—Representative histograms of flow cytometry analysis of EF5 intensity in isolated bone marrow and spleen cells from mice treated without or with EF5. EAC: euthanized animal control exposed to complete anoxia. FIG. 1D—Quantification of EF5+ and EF5− cell frequencies in the bone marrow (N=10) and spleen (N=5). FIG. 1E-Representative image of EF5 immunofluorescent staining in murine bone marrow.

FIGS. 2A-2D are graphs depicting EF5 measurement of bone marrow and fracture gap intracellular oxygenation. FIG. 2A-Cells from osteotomized (GAP: fracture gap; iBM: ipsilateral bone marrow) and un-injured bones (cBM: contralateral bone marrow) were isolated and analyzed for EF5 frequency and representative histograms. FIGS. 2B-2D—Quantification of EF5+ cells (3, 5 and 7 days post-fracture-dpf) shown normalized to cBM (N=5-6). Statistical analysis: Mann-Whitney test.

FIG. 3 is a graph depicting direct measurement of contralateral bone marrow and fracture gap tissue oxygen tension (mmHg) using the Oxyphor method, a metalloporphyrin whose phosphorescence decays at a rate proportional to the local oxygen tension. This allowed measurement of objective oxygen levels in tissues. Mice were injected with Oxyphor PtG4 one day prior to surgery. Osteotomized (GAP: fracture gap), and un-injured bones (cBM: contralateral bone marrow) were used for in vivo lifetime measurement of phosphorescence quenching rate in the presence of oxygen; results are displayed as absolute pO2 measurement (mmHg; N=4-5). Statistical analysis: Mann-Whitney.

FIGS. 4A and 4B are graphs depicting flow cytometry identification of CD45+ Ter119+ erythroid progenitor cells in the fracture gap at 3dpf. Contralateral bone marrow (cBM, FIG. 4A) and fracture gap (GAP, FIG. 4B) feature 5.8% and 45.5% CD45+ Ter119+ cells (indicated by arrow) at 3 dpf. Representative of N=5/group.

FIGS. 5A-5E are a diagram depicting workflow (FIG. 5A) and graphs depicting single cell RNA-sequencing of fracture hematoma under stiff fixation and contralateral bone marrow (FIGS. 5B-5E). FIG. 5B—UMAP of all cells. FIG. 5C—UMAP of erythroid progenitor cells. FIG. 5D—Dot plot selected genes. FIG. 5E—relative cumulative cell frequency of cluster 2, 4, and 6.

FIGS. 6A and 6B are graphs depicting the effects of CD71 blocking antibody on erythrocyte precursors and oxygenation in the gap. FIG. 6A—EF5 histograms showing shift to EF5+ (hypoxic) cells after αCD71 Ab treatment at dpf. FIG. 6B—Effect of αCD71 Ab on tissue oxygenation, measured by Oxyphor. N=3-6 per group.

FIGS. 7A-7D are graphs depicting the effects of CD71 blocking antibody on fracture repair. FIG. 7A—MicroCT imaging of bone formation. N=8/group. FIG. 7B—Quantification of bone formation in the treated groups. FIG. 7C—Immunostaining for Osterix (osteoblast-lineage) and Endomucin (endothelial cells). Anti-CD71 Ab significantly reduces the variability in the fracture healing outcome—all fractures heal similar (N=8/group). White area=newly formed bone. FIG. 7D-Change in bone volume (BV) over total volume (TV) at 14 dpf after treatment with isotype (Iso) or αCD71 antibody. Bar graphs show mean±SEM and individual data points. Statistical analysis: Unpaired t-test.

FIGS. 8A-8F are boxplots showing mRNA expression levels of selected genes involved in hemoglobin formation. Significant gene expression differences were identified using Marker Sequential Test (MAST) with Bonferroni corrections. FIG. 8A—mRNA expression of Tfrc; FIG. 8B—mRNA expression of Hba-a1; FIG. 8C—mRNA expression of Alad; FIG. 8D—mRNA expression of Uros; FIG. 8E—mRNA expression of Ppox; and FIG. 8F-mRNA expression of Fech.

FIGS. 9A and 9B depict the effect of a partially biodegradable scaffold implanted into the fracture site. FIG. 9A is a photograph of the implanted GelMa/firbin scaffold in the fracture site immediately after surgery (left image) and 14 dpf (right image) and FIG. 9B is a histology staining (Movat's Pentachrome staining) of a bone fracture gap with an implanted partially biodegradable GelMa/fibrin scaffold.

FIGS. 10A-10C are images from a histology staining of bone fracture gap with an implanted biodegradable fibrin scaffold. FIG. 10A-3D reconstruction of microCT image. FIG. 10B-Histological staining (Movat's Pentachrome staining) of fracture site 14 dpf. FIG. 10C-Immunostaining for Osterix (osteoblast-lineage; white) and Endomucin (endothelial cells; magenta).

FIGS. 11A and 11B are diagrams depicting different scenarios of scaffold implantation and seeding for a method of enhancing bone fracture healing at a bone fracture site in a subject.

### DETAILED DESCRIPTION OF ILLUSTRATIVE EMBODIMENTS

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, certain preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of +20% or +10%, more preferably +5%, even more preferably =1%, and still more preferably +0.1% from the specified value, as such variations are appropriate to perform the disclosed methods.

As used herein, “increase” or “enhance” refers to a change in the value of a given variable that is at least 10% or more, for example, 20%, 30%, 40%, or 50%, 60%, 70%, 80%, 90% or more higher, and/or 1.1 fold, 1.2 fold, 1.4 fold, 1.6 fold, 1.8 fold, 2.0 fold or more higher, and any and all whole or partial increments therebetween, than a control reference value.

As used herein, “decrease” or “reduce” refers to a change in the value of a given variable that is at least 10% lower or more, for example, 20%, 30%, 40%, or 50%, 60%, 70%, 80%, 90% lower or more, and/or 1.1 fold, 1.2 fold, 1.4 fold, 1.6 fold, 1.8 fold, 2.0 fold or more lower, and any and all whole or partial increments in between, than a control reference value.

As used herein, the terms “control,” or “reference” can be used interchangeably and refer to a value that is used as a standard of comparison.

The term “heal” or “repair” as used within the context of the present invention is meant to include therapeutic treatment for the disease or disorder. As used herein, the term “heal” or “repair” and associated terms such as “treat” and “treating” means the reduction of the progression, severity and/or duration of a disease condition or at least one symptom thereof. In the context of bone fracture healing or repair, the terms “heal” or “repair” is meant to refer to the increase in bone volume at the fracture site, filling of the fracture site with bone tissue, union at the fracture site, and/or an improvement in at least one measurable symptom of bone fracture.

As used herein, the term “composition” refers to at least one compound useful within the invention. The composition can be a small molecule, a hormone, a growth factor, a nucleic acid, an enzymatic protein, a chemokine, a cytokine, a structural protein or peptide, an extracellular matrix protein or peptide, a binding protein or peptide (such as an antibody or an antibody fragment, a receptor or a receptor fragment), a cell, a cell fraction, or any combination thereof.

A “subject” or “patient,” as used therein, can be a human or non-human mammal. Non-human mammals include, for example, livestock and pets, such as ovine, bovine, porcine, canine, feline and murine mammals. Preferably, the subject is a human. Ranges: throughout this disclosure, various aspects of the invention can be presented in a range format.

It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Device for Bone Fracture or Segmental Defect Repair

Provided herein are devices for bone fracture or segmental defect repair. The devices can comprise: an implantable substrate; a first active composition, the first active composition optionally in contact with the implantable substrate; and a second active composition, the second active composition optionally in contact with the implantable substrate, wherein the first active composition comprises one or more compositions for bone union, and wherein the second active composition comprises one or more oxygen-modulating compositions.

The devices can comprise implantable substrates, such as any one or more of an orthopedic implant, a scaffold, a gel, a putty, a gel and putty, granules, a sponge, a foam, demineralized bone matrix particles, fibers, flexible strips, a graft, an injectable bone graft substitute, a collection of microparticles, or a collection of nanoparticles. The implantable substrates can comprises a biocompatible material. The implantable substrates can comprises a biodegradable or non-biodegradable material. In some embodiments, the device comprises an implantable substrate, wherein the implantable substrate is a scaffold.

In some embodiments, the implantable substrate comprises a material selected from the group consisting of polylactic acid, polyglycolic acid, PLGA polymers, alginates and alginate derivatives, gelatin, collagen, fibrin, hyaluronic acid, hydroxyapatite, calcium phosphate, tri-calcium phosphate, demineralized bone matrix, calcium sulfate, collagen matrix, laminin-rich gels, agarose, natural and synthetic polysaccharides, polyamino acids, polypeptides, polyesters, polyanhydrides, polyphosphazines, poly(vinyl alcohols), poly(alkylene oxides), poly(allylamines) (PAM), poly(acrylates), modified styrene polymers, pluronic polyols, polyoxamers, poly(uronic acids), poly(vinylpyrrolidone), and copolymers or graft copolymers thereof.

The implantable substrate can comprise a coating. The implantable substrate can comprise cells. The implantable substrate can be coated or seeded with at least one of the first active composition and the second active composition.

The first active composition can comprise any one or more of a cell, an extracellular matrix protein, a growth factor, a cytokine, or a chemoattractant. For example, the first active composition can comprise any one or more of erythropoietin (Epo), stem cell factor (SCF), bone morphogenetic protein 4 (BMP-4), or growth/differentiation factor 15 (GDF-15).

The second active composition can comprise any one or more of antibodies, hypoxia inducing factor (HIF) stabilizers, transferrin blocking agents, iron metabolism modulators, hemoglobin inhibitors, or iron chelators. For example, the second active composition can comprise any one or more of anti-cluster differentiation-71 (anti-CD-71) antibody, anti-CD-71 fragment antigen-binding (Fab), anti-CD-71 single chain variable fragment (scFv), anti-CD-71 diabody, or an iron chelator.

In some embodiments, the second active composition of the device modulates oxygen levels at a bone fracture site following device implantation at the site. The oxygen levels at the site of implantation of the device may be increased or decreased.

In some embodiments, the oxygen levels at the site of implantation of the device are increased. The increase in oxygen levels may be a change of at least 10% or more, for example, 20%, 30%, 40%, or 50%, 60%, 70%, 80%, 90% or more higher, and/or 1.1 fold, 1.2 fold, 1.4 fold, 1.6 fold, 1.8 fold, 2.0 fold or more higher, and any and all whole or partial increments therebetween, than a control reference oxygen level. A control reference oxygen level may be a level at a bone fracture site without the implanted device, or with an implanted device lacking the second active composition.

In some embodiments, the oxygen levels at the site of implantation of the device are decreased. The decrees in oxygen levels may be a change of at least 10% or more, for example, 20%, 30%, 40%, or 50%, 60%, 70%, 80%, 90% or more lower, and/or 1.1 fold, 1.2 fold, 1.4 fold, 1.6 fold, 1.8 fold, 2.0 fold or more lower, and any and all whole or partial increments therebetween, than a control reference oxygen level. A control reference oxygen level may be a level at a bone fracture site without the implanted device, or with an implanted device lacking the second active composition.

A control reference oxygen level may be a level of oxygen at an ipsilateral site, at a contralateral site, at a bone fracture site without the implanted device, or with an implanted device lacking the second active composition.

### Methods of Enhancing Healing at Bone Fracture or Segmental Defect Sites

Also provided are methods of enhancing healing at bone fracture or segmental defect sites in subjects. The methods can comprise administering at the bone fracture or segmental defect sites a device comprising: an implantable substrate; a first active composition, the first active composition optionally in contact with the implantable substrate; and a second active composition, the second active composition optionally in contact with the implantable substrate, wherein the first active composition comprises one or more compositions for bone union, and wherein the second active composition comprises one or more oxygen-modulating compositions.

The methods can comprise administering the devices at the bone fracture sites at a time of surgery.

The disclosed methods can enhance healing of bone fractures comprising any one or more of a fracture or segmental defect of long bone, a fracture or segmental defect of a rib, or a fracture or fusion of vertebrae, an osteoporotic fracture, or a mandibular defect.

In some embodiments, the implantable substrate of the device is seeded with autologous or allogeneic bone marrow cells.

The methods can comprise reducing an oxygenation level at the bone fracture site after administering the device.

The methods can comprise at least one of decreasing tissue oxygen tension or increasing a number of hypoxic cells at the bone fracture site.

The methods can comprise increasing osteoprogenitor activation and angiogenesis at the bone fracture or segmental defect site.

The methods can comprise enhancing bone fracture or segmental defect healing by increasing bone volume at the bone fracture site.

In some embodiments, enhancing bone fracture or segmental defect healing comprises achieving bone volume increase of up to about 10%, about 20%, about 30%, about 40%, about 50%, about 60%, about 70%, about 80%, about 90%, or up to about 100% relative to a control, or relative to the result of standard procedures, in at least 90% of bone fracture or segmental defect sites with the administered device.

In some embodiments, enhancing bone fracture or segmental defect healing comprises reducing time to union at the bone fracture or segmental defect site.

Also provided are methods of reducing incidences of non-union among subjects with bone fractures. The methods of reducing incidences of non-union can comprise administering at the bone fracture or segmental defect sites a device comprising: an implantable substrate; a first active composition, the first active composition optionally in contact with the implantable substrate; and a second active composition, the second active composition optionally in contact with the implantable substrate, wherein the first active composition comprises one or more compositions for bone union, wherein the second active composition comprises one or more oxygen-modulating compositions, and wherein the incidence of non-union at the bone fracture or segmental defect site is reduced relative to the incidence in the control subjects, or relative to the incidence after standard procedures, among the subjects. The incidence of non-union at the bone fracture or segmental defect site can be reduced to below about 15%, below about 14%, below about 13%, below about 12%, below about 11%, below about 10%, below about 9%, below about 8%, below about 7%, below about 6%, below about 5%, below about 4%, below about 3%, below about 2%, or below about 1% relative to the incidence in the control subjects, or relative to the incidence after standard procedures, among the subjects.

The present invention relates to a device designed for guiding the recruitment, enrichment, and differentiation of erythroid lineage cells, with the additional capability of modulating the function of these cells to influence the oxygen environment in the surrounding tissue. The device may comprise a scaffold that incorporates or is coated with two bioactive compositions, providing a versatile platform for potential therapeutic applications in the field of regenerative medicine.

The oxygen modulation device can comprise a scaffold designed for use in vivo or in vitro, allowing for the recruitment and manipulation of erythroid progenitor cells, which trap and release oxygen using the protein hemoglobin. The scaffold is composed of biocompatible materials, which may be either biodegradable or resistant to degradation, providing flexibility in application. Examples of potential scaffold compositions include polylactic acid, polyglycolic acid, PLGA polymers, alginates and alginate derivatives, gelatin, collagen, fibrin, hyaluronic acid, hydroxyapatite, calcium phosphate, tri-calcium phosphate, demineralized bone matrix, calcium sulfate, collagen matrix, laminin-rich gels, agarose, natural and synthetic polysaccharides, polyamino acids, polypeptides, polyesters, polyanhydrides, polyphosphazines, poly(vinyl alcohols), poly(alkylene oxides), poly(allylamines) (PAM), poly(acrylates), modified styrene polymers, pluronic polyols, polyoxamers, poly(uronic acids), poly(vinylpyrrolidone), and copolymers or graft copolymers of any of the above.

In some embodiments, the disclosure provides devices with substrates that incorporate in or are coated with two distinct bioactive compositions. The first bioactive composition, responsible for attraction and enrichment, includes growth factors or other relevant proteins such as Epo, SCF, BMP-4, and GDF-15. The second bioactive composition focuses on functional modification of oxygen at the device implantation site, and may involve CD71 antibodies, other transferrin blocking agents, iron metabolism modulators, HIF stabilizers, or hemoglobin (formation) targeting compounds.

The disclosed devices function by guiding the recruitment, enrichment, and differentiation of erythroid progenitor cells, while simultaneously modulating their function to regulate the oxygen environment in the surrounding tissue. This dual functionality enhances the therapeutic potential of the device for applications such as tissue regeneration, bone or wound healing, and treatment of disorders related to oxygen availability.

The seeding of the substrate can occur in various ways, either in vivo or in vitro. For instance, cells may be recruited after transplantation to migrate into the device or injected directly into the scaffold. Alternatively, cells can be seeded onto the substrate by incubating the substrate in a solution containing the cells, providing adaptability to different therapeutic scenarios.

The disclose devices represent a dual function approach to tissue engineering and regenerative medicine. The incorporation of dual bioactive compositions on a biocompatible scaffold offers a versatile platform for manipulating erythroid progenitor cells and modulating the local tissue oxygen environment.

### EMBODIMENTS

Provided below are exemplary enumerated embodiments. The following list of embodiments is intended to complement, rather than displace or supersede, the previous descriptions. Any part of any Embodiment can be combined with any part or parts of any other one or more Embodiments.

Embodiment 1. A device for bone fracture or segmental defect healing, comprising: an implantable substrate; a first active composition, the first active composition optionally in contact with the implantable substrate; and a second active composition, the second active composition optionally in contact with the implantable substrate, wherein the first active composition comprises one or more compositions for bone healing, and wherein the second active composition comprises one or more oxygen-modulating compositions.

Embodiment 2. The device of Embodiment 1, wherein the implantable substrate is any one or more of an orthopedic implant, a scaffold, a gel, a putty, a gel and putty, granules, a sponge, a foam, demineralized bone matrix particles, fibers, flexible strips, a graft, an injectable bone graft substitute, a collection of microparticles, or a collection of nanoparticles.

Embodiment 3. The device of Embodiment 1 or 2, wherein the implantable substrate comprises a biocompatible material.

Embodiment 4. The device of any one of Embodiments 1-3, wherein the implantable substrate comprises a biodegradable or non-biodegradable material.

Embodiment 5. The device of any one of Embodiments 1-4, wherein the implantable substrate is a scaffold.

Embodiment 6. The device of any one of Embodiments 1-5, wherein the implantable substrate comprises a material selected from the group consisting of polylactic acid, polyglycolic acid, PLGA polymers, alginates and alginate derivatives, gelatin, collagen, fibrin, hyaluronic acid, hydroxyapatite, calcium phosphate, tri-calcium phosphate, demineralized bone matrix, calcium sulfate, collagen matrix, laminin-rich gels, agarose, natural and synthetic polysaccharides, polyamino acids, polypeptides, polyesters, polyanhydrides, polyphosphazines, poly(vinyl alcohols), poly(alkylene oxides), poly(allylamines) (PAM), poly(acrylates), modified styrene polymers, pluronic polyols, polyoxamers, poly(uronic acids), poly(vinylpyrrolidone), and copolymers or graft copolymers thereof.

Embodiment 7. The device of any one of Embodiments 1-6, wherein the implantable substrate is coated or seeded with at least one of the first active composition and the second active composition.

Embodiment 8. The device of any one of Embodiments 1-7, wherein the first active composition comprises any one or more of a cell, an extracellular matrix protein, a growth factor, a cytokine, or a chemoattractant.

Embodiment 9. The device of any one of Embodiments 1-8, wherein the first active composition comprises any one or more of erythropoietin (Epo), stem cell factor (SCF), bone morphogenetic protein 4 (BMP-4), or growth/differentiation factor 15 (GDF-15).

Embodiment 10. The device of any one of Embodiments 1-9, wherein the second active composition comprises any one or more of antibodies, hypoxia inducing factor (HIF) stabilizers, transferrin blocking agents, iron metabolism modulators, hemoglobin inhibitors, or iron chelators.

Embodiment 11. The device of any one of Embodiments 1-9, wherein the second active composition comprises any one or more of anti-cluster differentiation-71 (anti-CD-71) antibody, anti-CD-71 fragment antigen-binding (Fab), anti-CD-71 single chain variable fragment (scFv), anti-CD-71 diabody, or an iron chelator.

Embodiment 12. A method of enhancing healing at a bone fracture site in a subject, comprising: administering at the bone fracture site a device according to any one of Embodiments 1-11.

Embodiment 13. The method of Embodiment 12, comprising administering the device at the bone fracture site at a time of surgery.

Embodiment 14. The method of Embodiment 12 or 13, wherein the bone fracture comprises any one or more of a fracture or segmental defect of long bone, a fracture or segmental defect of a rib, a fracture or fusion of vertebrae, an osteoporotic fracture, or a mandibular defect.

Embodiment 15. The method of any one of Embodiments 12-14, wherein the implantable substrate is seeded with autologous or allogeneic bone marrow cells.

Embodiment 16. The method of any one of Embodiments 12-15, wherein an oxygenation level at the bone fracture or segmental defect site is reduced after administering the device.

Embodiment 17. The method of any one of Embodiments 12-16, wherein the enhancing of bone fracture or segmental defect healing comprises at least one of decreasing tissue oxygen tension or increasing a number of hypoxic cells at the bone fracture or segmental defect site.

Embodiment 18. The method of any one of Embodiments 12-17, wherein the enhancing of bone fracture or segmental defect healing comprises an increase in osteoprogenitor activation and angiogenesis at the bone fracture or segmental defect site.

Embodiment 19. The method of any one of Embodiments 12-18, wherein the enhancing of bone fracture or segmental defect healing comprises an increase in bone volume at the bone fracture or segmental defect site.

Embodiment 20. The method of any one of Embodiments 12-19, wherein the enhancing of bone fracture or segmental defect healing comprises achieving union in at least 90% of bone fracture or segmental defect sites with the administered device.

Embodiment 21. The method of any one of Embodiments 12-20, wherein the enhancing of bone fracture or segmental defect healing comprises reducing time to union at the bone fracture or segmental defect site.

Embodiment 22. A method of reducing incidence of non-union among subjects with bone fracture, comprising: administering the device of any one of Embodiments 1-11 at the bone fracture site of each subject, wherein the incidence of non-union at the bone fracture site is reduced to below 5% among the subjects.

### EXAMPLES

### Example 1. Measurement of Intracellular Oxygenation by EF5

Intracellular oxygen levels of bone marrow cells cultured under defined oxygen conditions in vitro were studied using EF5. EF5 is a nitroimidazole, that is selectively reduced by nitroreductase enzymes under hypoxic conditions, resulting in the formation of EF5 adducts that can be visualized with fluorophore-coupled antibody (16). Orthogonal methods for in vivo measurements of oxygen levels were established. First, EF5 was used for staining. Second, oxygen levels were measured by the phosphorescence quenching method using an established probe Oxyphor PtG4 (17) (as detailed in Example 2).

### Materials and Methods

EF5 labeling of bone marrow cells under defined oxygen conditions (0.1%-10% pO2) in vitro was carried out with 100 μM EF5.

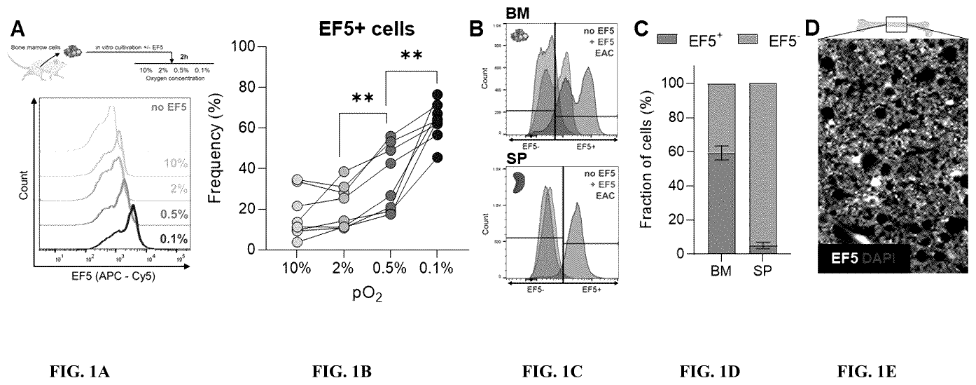

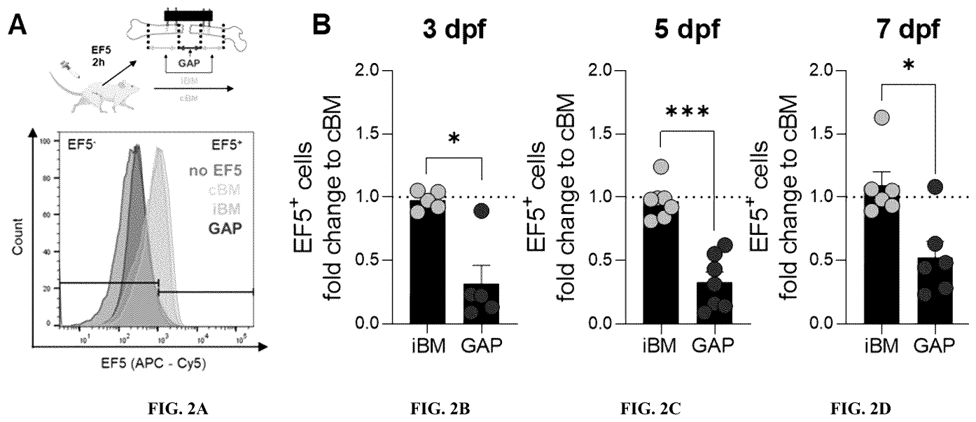

EF5 labeling of bone marrow cells in vivo was also carried out (FIG. 1C). Briefly, 12-14 week old C57BL/6J female mice were injected with 10 mM EF5 compound. As a positive control for in situ EF5− labeling of marrow cells in low oxygen conditions, mice were injected with EF5 and euthanized after 30 min. The body was then kept at 37° C. in entirely anoxic conditions for 45 min (i.e., euthanized animal control, EAC). As a further control, EF5 staining of the spleen (which is a known oxygen reservoir with high oxygen tension of 8-10% pO2) was performed. To control for nonspecific background staining, non-EF5 treated controls (EF5−) were evaluated. A BD FACSCanto II was used for flow cytometry and data was analyzed using FlowJo software. For immunofluorescence staining, sections were fixed in 4% paraformaldehyde, blocked with 5% goat serum, and stained 2 h with Cy3 conjugated anti-EF5 antibody. More specifically, all procedures were conducted in accordance with UPenn IACUC regulations. Eye ointment, clindamycin, sustained-release buprenorphine (ZooPharm; s.c.; 1 mg/kg) and NaCl were applied during the preparation. The left femur was bluntly exposed, and the pins were placed though the connector bar of the external fixator laterally parallel to the femur. A 0.5 mm gap was created between the middle pins using a Gigli wire. For EF5 staining (3 days post-fracture-dpf): Mice were injected with 10 mM EF5 compound and euthanized after 2 hours. Cells were isolated from the fracture gap (FIG. 2A; GAP); the adjacent bone marrow of the same limb (FIG. 2A; iBM) and the bone marrow from the uninjured contralateral bone (FIG. 2A; cBM) and fixed with 4% paraformaldehyde before staining overnight with Cy5-conjugated anti-EF5 antibody (clone: ELK3-51). A BD FACSCanto II was used for flow cytometry and data were analyzed using FlowJo software. EF5 labeling of haematoma cells in the early bone fracture gap in vivo was also carried out. For this study, 0.5 mm osteotomies were performed in femora of C57BL/6J female mice aged 12-16 weeks and stabilized with stiff external fixation plates (MouseExFix, RISystem). EF5 injection, staining, and flow cytometry was performed as above at 3, 5, and 7 days post-fracture (dpf). Cells were isolated from three regions of interest: the fracture gap (GAP); the ipsilateral bone marrow of the same limb (iBM) and the bone marrow from the uninjured contralateral bone (cBM).

### Results

EF5 staining in cultured bone marrow cells increased monotonically with decreasing environmental oxygen tension from 10% to 0.1% pO2 (FIG. 1A). Together, these data establish that EF5 effectively marks intracellular hypoxia in vitro.

EF5 staining in murine bone marrow confirmed intracellular oxygen heterogeneity of bone marrow cells in vivo (FIGS. 1C and 1D). Enforcing anoxia (EAC) in the bone marrow resulted in a complete shift of cells. Moreover, no EF5 positive cells were found in the spleen under normal conditions, although anoxia resulted in 100% EF5 positivity in spleen cells. Immunofluorescence staining of the whole bone marrow identified regions with high and low EF5 signal intensity, confirming heterogenous intracellular hypoxia in situ (FIG. 1E).

The ipsilateral and contralateral bone marrow exhibited about 60% EF5 positive cells, as expected. However, only a small fraction of cells in the day 3 fracture hematoma were EF5+ (FIGS. 2A and 2B). Rather the fracture gap cells exhibited similar oxygenation to the native spleen (FIG. 2B). This demonstrates that, contrary to persistent dogma, the cells of the early fracture gap are not hypoxic. Further, the frequency of EF5+ (hypoxic) cells increased from day 3 to 5 to 7 after surgery but did not reach the level of cellular hypoxia characteristic of the un-injured bone marrow by day 7 (FIGS. 2C and 2D). As before, a fully anoxic euthanized animal control (EAC) was also tested. 95% of fracture gap cells were EF5+ in EAC (N=3; data not shown).

### Example 2. Direct Measurement of Tissue Oxygen Tension by Oxyphor PtG4

### Materials and Methods

As an orthogonal approach, tissue oxygen levels were directly measured in bone marrow and early bone fracture hematoma in vivo using the direct oxygen tension probe, Oxyphor PtG4. Briefly, lifetime imaging of Oxyphor PtG4 phosphorescence quenching by O₂ was performed. Oxyphor PtG4 was injected in the tail vein 1 day prior to surgery to allow for systemic and even distribution in the fracture hematoma and gap after osteotomy. Osteotomy surgery was performed as described above. At 3, 7 and 14 dpf, mice were anesthetized with isoflurane and the osteotomized and contralateral bones were carefully dissected and exposed. Oxygen levels were measured using a fiber-optic phosphorimeter.

### Results

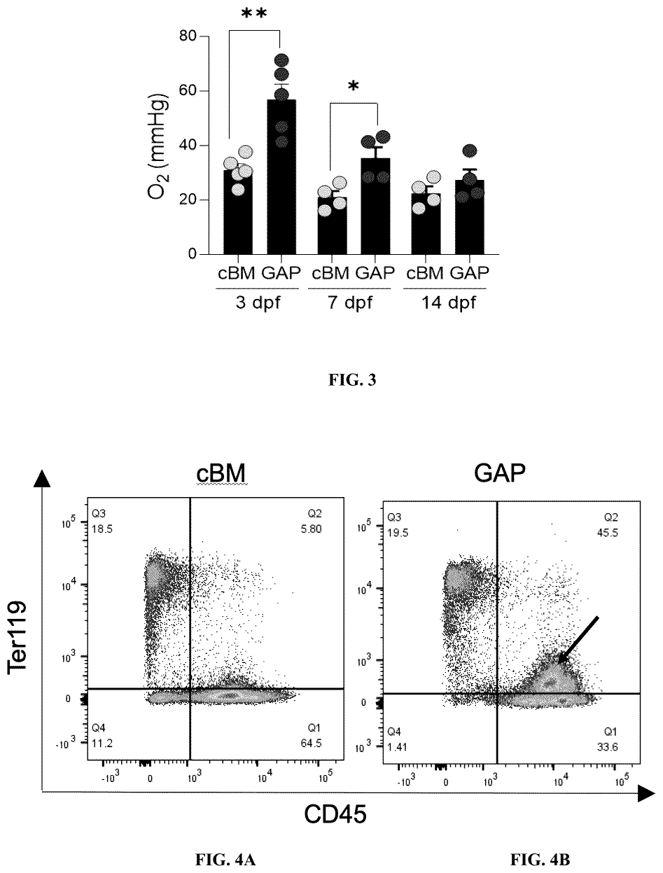

Consistent with the EF5 data, tissue oxygen measurement demonstrated that the fracture hematoma is not hypoxia, but rather exhibits oxygen levels commensurate with those of the spleen (56.51±14.44 mmHg; equivalent to ˜8% pO2) (FIG. 3). In contrast, the oxygen tension of the contralateral bone marrow was measured at 32.78±3.7 mmHg; equivalent to ˜3% pO2, consistent with previously reported values.

Together, these two orthogonal methods show that, in contrast to the prior consensus assumption in the field, the early fracture gap features high initial oxygenation, which drops to approach bone marrow hypoxia levels by 14 dpf.

### Example 3. Fracture Induces Local Erythropoiesis

Next, it was investigated as to why the fracture gap exhibited such high oxygen levels.

### Materials and Methods

The cells of the fracture haematoma were flow-sorted for Ter119+ cells, which mark erythrocytes.

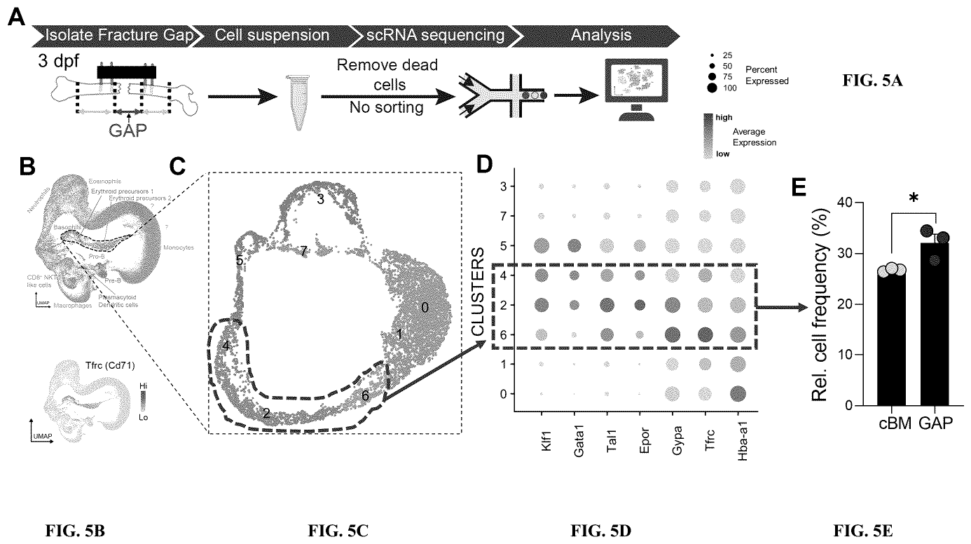

To delineate the different erythroid progenitor cell populations, single cell RNA-sequencing was performed. Cells were isolated from the fracture gap at 3 dpf from female C57/B16J mice (N=3), 14-16 weeks of age, with stiff fixators. Un-injured contralateral bone marrow was also isolated. Next-generation sequencing scRNA-seq using the 10× Genomics Chromium platform was performed (FIG. 5A).

### Results

It was found that substantial numbers of mature erythrocytes were in the fracture gap. However, by serendipity, it was also observed that a distinct population of Ter119-low, CD45-high, and CD71-high erythrocyte precursors were present (FIGS. 4A and 4B). These CD71+ erythrocyte precursor cells bloomed in large numbers locally in the fracture gap by 3 days post-fracture but were not detectable in either the ipsilateral or contralateral bone marrow.

Louvain clustering revealed 21 clusters, which were identified as composing erythrocyte precursors, macrophages, and others (FIG. 5B). Through Gene set enrichment analysis (GSEA), a subset of erythrocyte precursors was found (FIG. 5B). The erythrocyte precursors were identified as predominately expressing Tfrc (CD71; FIG. 5B). We further identified 8 subpopulations of erythroid progenitor cells in the fracture hematoma (FIG. 5C). Relevant gene expression analysis (FIG. 5D) and cell frequency analyses identified a subgroup of progenitor cells being enriched in the fracture gap when compared to the contralateral bone marrow (FIG. 5E).

### Example 4. Targeting CD71+ Cells for Fracture Repair

### Materials and Methods

CD71 is also known as “Transferrin receptor 1.” CD71 is a marker for erythrocyte precursors, and is required for iron import into erythroid cells to enable formation of hemoglobin, an iron-containing protein that binds oxygen. Two preliminary studies were performed. First, fractures were treated with anti-CD71 blocking antibody (or Isotype IgG control), and intracellular oxygen and erythroid-lineage cells in the fracture gap were quantified at day 3 post-fracture. Second, the consequences of CD71 blockade on bone repair was evaluated by microCT and histology at 14 dpf. More specifically, either a monoclonal rat anti-mouse CD71 antibody (clone: 8D3; 100 μg) or a rat IgG2a isotype control was injected in the fracture gap during surgery. EF5 staining and Oxyphor measurement were performed at 3 dpf as described above. MicroCT analysis (Scanco μCT 45) and histology/immunofluorescence (H&E; endomucin−Emcn=vessel; osterix−Osx=osteoblasts) were performed at 14 dpf. Statistical analysis was performed using ANOVA/Tukey's and Student's t-test, as appropriate. An scRNA-seq was performed to determine the mechanism of CD71 blockade.

### Results

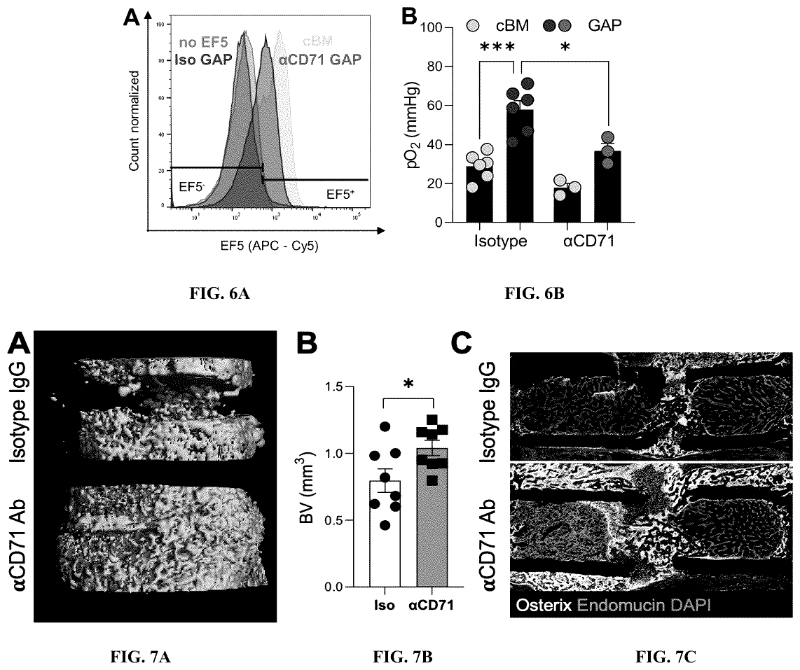

It was found that injection of CD71 blocking antibody (100 μg) in the fracture gap at the time of surgery increased the fraction of EF5− positive cells, indicating greater degree of cellular hypoxia in the fracture gap (FIGS. 6A and 6B).

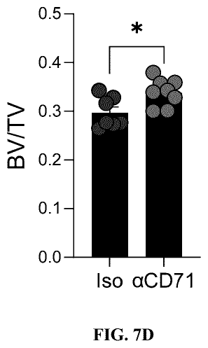

Strikingly, CD71 blockade significantly increased bone formation and angiogenesis in the callus and fracture gap (FIGS. 7A-7D). Anti-CD71 Ab significantly reduces the variability in the fracture healing outcome-all fractures heal similar (N=8/group). White area=newly formed bone.

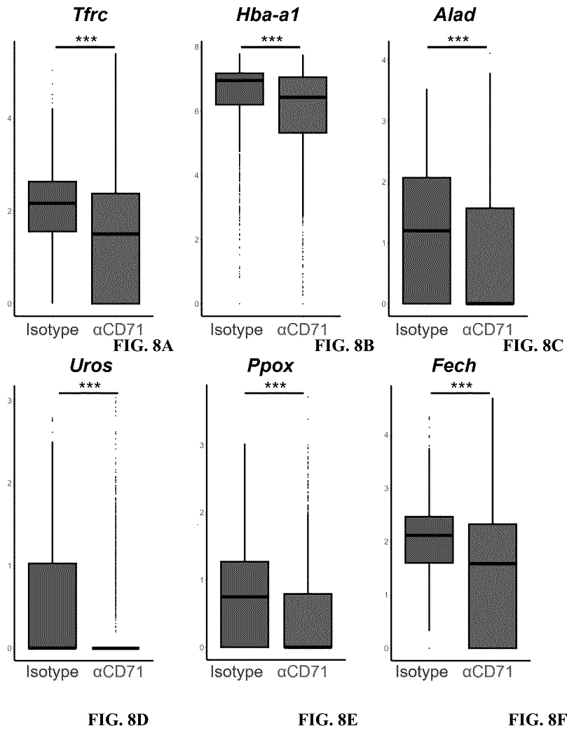

Comparing mRNA expression of erythroid progenitors from fractured bones treated with and without anti-mouse CD71 antibody, we found significantly lower expression of genes essential for hemoglobin formation following CD71 blockade. Restricting iron import reduces hemoglobin formation capacity, thereby lowering oxygen-binding capability, which supports the mechanism underlying our findings (FIGS. 8A-8F).

### Example 5. Implantation of Scaffolds at Bone Fracture Gaps

### Materials and Methods

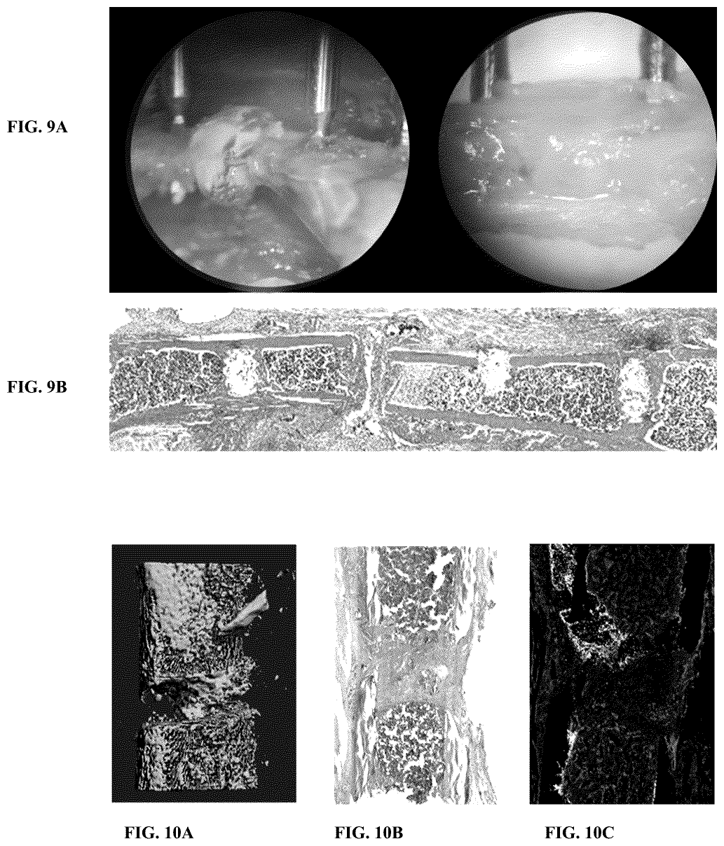

To show safety and feasibility, two different scaffold types, biodegradable and partially biodegradable, were implanted into the fracture gap in mice femurs. As partially biodegradable scaffold GelMA/fibrin was used consisting of Gelatin methacrylate (GelMA; 5% w/w) and 5 mg/ml fibrinogen mixed in a 0.2% w/w lithium phenyl-2,4,6-trimethylbenzoylphosphinate (LAP) solution to induce crosslinking (all products were purchased from Cellink, Gothenburg, Sweden). As a fully biodegradable scaffold human recombinant fibrin (5 mg/ml) was used. The bone fractures were created as detailed in Example 1 and the scaffolds were placed in the fracture gap (site; FIG. 9A). Healing outcomes were measured using histology and microCT at 14 dpf.

### Results

Both scaffold types integrated into the fracture site and allowed for new tissue formation (FIG. 9B and FIGS. 10A-10C). The scaffold implantation did not induce extensive immune reaction, as evidenced by histology and immunostaining for macrophages, and the scaffolds allowed colonization by endogenous cells, labeled by the nuclear stain, DAPI (FIGS. 10B-10C)

FIGS. 9A and 9B depict the effect of a partially biodegradable scaffold implanted into the fracture site. FIG. 9A is a photograph of the implanted GelMa/firbin scaffold in the fracture site immediately after surgery (left image) and 14 dpf (right image) and FIG. 9B is a histology staining (Movat's Pentachrome staining) of a bone fracture gap with an implanted partially biodegradable GelMa/fibrin scaffold.

FIGS. 10A-10C are images from a histology staining of bone fracture gap with an implanted biodegradable fibrin scaffold. FIG. 10A-3D reconstruction of microCT image. FIG. 10B-Histological staining (Movat's Pentachrome staining) of fracture site 14 dpf. FIG. 10C-Immunostaining for Osterix (osteoblast-lineage; white) and Endomucin (endothelial cells; magenta).

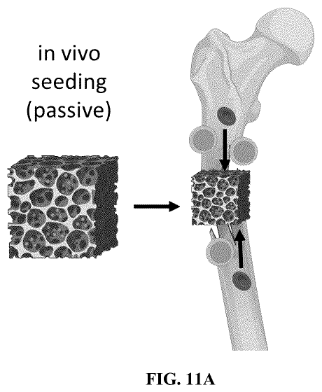

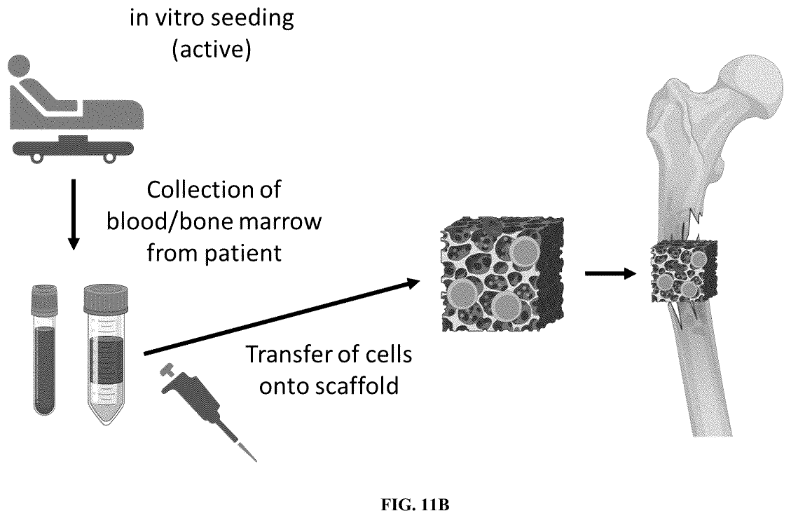

FIGS. 11A and 11B present the different scenarios of scaffold implantation and seeding for a method of enhancing bone fracture healing at a bone fracture site in a subject. The seeding of the scaffold can occur in various ways, either in vivo or in vitro. For instance, cells may be recruited after transplantation to migrate into the device (or injected directly into the scaffold). Alternatively, cells can be seeded onto the scaffold by incubating the scaffold in a solution containing the cells, providing adaptability to different therapeutic scenarios.

As noted above, it was discovered that in bone fractures in which the bone marrow is injured, this injury causes activation of a special type of blood stem cells: erythroid progenitor cells (the precursors to red blood cells). As they mature, these cells express hemoglobin, which concentrates oxygen at the fracture site, resulting in elevated oxygen tension and impaired and highly variable healing outcomes due to suppressed recruitment of pro-regenerative neovascularization and osteoprogenitor invasion.

A therapeutic strategy is proposed that addresses these needs without these limitations. Specifically, the device features a matrix for delivery, a chemotactic factor that concentrates or traps local erythroid progenitor cells into the scaffold, and an agent that prevents erythropoietic maturation. This device would be implanted at the fracture site at the time of surgery and would prevent erythroid progenitor cells from concentrating oxygen at the fracture site, producing a hypoxic environment to stimulate blood vessel invasion and osteoprogenitor co-mobilization.

The experimental evidence demonstrate that the devices can deliver chemotactic agents to bone fractures using a biodegradable scaffold that mimics the composition of the natural fracture hematoma. It is shown that delivery of a CD71 blocking antibody, which blocks the iron transporter, Transferrin Receptor 1, blocks local erythropoiesis, induces fracture gap hypoxia, and stimulates bone formation. Importantly, treatment with the CD71 blocking antibody not only increased the mean amount of bone formed, but also dramatically reduced the inter-sample variability of both blood vessel and osteoprogenitor invasion. This is particularly important because typical treatments that increase mean bone formation also increase variability. The disclosed compositions and methods would be transformative for the treatment of fractures because they reduce patient-to-patient variability and uncertainty of non-union during healing. Additionally, this is achieved without the side effects of a potent osteogenic growth factor.

### REFERENCES

[1] Schlundt C, Bucher C H, Tsitsilonis S, Schell H, Duda G N and Schmidt-Bleek K 2018 Clinical and Research Approaches to Treat Non-union Fracture Curr Osteoporos Rep 16 155-68
[2] Commission E 2004 European Action Towards Better Musculoskeletal Health.
[3] USA B a J I 2015 The Burden of Musculoskeletal Diseases in the United States (BMUS).
[4] Berrey B H, Jr., Lord C F, Gebhardt M C and Mankin H J 1990 Fractures of allografts. Frequency, treatment, and end-results The Journal of bone and joint surgery. American volume 72 825-33
[5] Cypher T J and Grossman J P 1996 Biological principles of bone graft healing J Foot Ankle Surg 35 413-7
[6] Ito H, Koefoed M, Tiyapatanaputi P, Gromov K, Goater J J, Carmouche J, Zhang X, Rubery P T, Rabinowitz J, Samulski R J, Nakamura T, Soballe K, O'Keefe R J, Boyce B F and Schwarz E M 2005 Remodeling of cortical bone allografts mediated by adherent rAAV-RANKL and VEGF gene therapy Nat Med 11 291-7
[7] Maes C, Kobayashi T, Selig M K, Torrekens S, Roth S I, Mackem S, Carmeliet G and Kronenberg H M 2010 Osteoblast precursors, but not mature osteoblasts, move into developing and fractured bones along with invading blood vessels Dev Cell 19 329-44
[8] Phelps E A and Garcia A J 2010 Engineering more than a cell: vascularization strategies in tissue engineering Curr Opin Biotechnol 21 704-9
[9] Hak D J, Fitzpatrick D, Bishop J A, Marsh J L, Tilp S, Schnettler R, Simpson H and Alt V 2014 Delayed union and nonunions: epidemiology, clinical issues, and financial aspects Injury 45 Suppl 2 S3-7
[10] Hankenson K D, Zmmerman G and Marcucio R 2014 Biological Perspectives of Delayed Fracture Healing Injury 45 S8-S15
[11] Gomez-Barrena E, Rosset P, Lozano D, Stanovici J, Ermthaller C and Gerbhard F 2015 Bone fracture healing: cell therapy in delayed unions and nonunions Bone 70 93-101
[12] Barcak E A and Beebe M J 2017 Bone Morphogenetic Protein: Is There Still a Role in Orthopedic Trauma in 2017? The Orthopedic clinics of North America 48 301-9
[13] Burks M V and Nair L 2010 Long-term effects of bone morphogenetic protein-based treatments in humans Journal of long-term effects of medical implants 20 277-93
[14] James A W, LaChaud G, Shen J, Asatrian G, Nguyen V, Zhang X, Ting K and Soo C 2016 A Review of the Clinical Side Effects of Bone Morphogenetic Protein-2 Tissue Eng Part B Rev 22 284-97
[15] Lad S P, Bagley J H, Karikari I O, Babu R, Ugiliweneza B, Kong M, Isaacs R E, Bagley C A, Gottfried O N, Patil C G and Boakye M 2013 Cancer After Spinal Fusion The Role of Bone Morphogenetic Protein Neurosurgery 73 440-9
[16] Koch C J 2002 Measurement of absolute oxygen levels in cells and tissues using oxygen sensors and 2-nitroimidazole EF5 Methods Enzymol 352 3-31
[17] Lebedev A Y, Cheprakov A V, Sakadzić S, Boas D A, Wilson D F and Vinogradov S A 2009 Dendritic phosphorescent probes for oxygen imaging in biological systems ACS Appl Mater Interfaces 1 1292-304
Swartz, H. M. et al. How best to interpret measures of levels of oxygen in tissues to make them effective clinical tools for care of patients with cancer and other oxygen-dependent pathologies. Physiol Rep 8, e14541, doi: 10.14814/phy2.14541 (2020).
Spencer, J. A. et al. Direct measurement of local oxygen concentration in the bone marrow of live animals. Nature 508, 269-273, doi: 10.1038/nature13034 (2014).
Carlier, A., Geris, L., van Gastel, N., Carmeliet, G. & Van Oosterwyck, H. Oxygen as a critical determinant of bone fracture healing—a multiscale model. J Theor Biol 365, 247-264, doi: 10.1016/j.jtbi.2014.10.012 (2015).
Claes, L., Recknagel, S. & Ignatius, A. Fracture healing under healthy and inflammatory conditions. Nat Rev Rheumatol 8, 133-143, doi: 10.1038/nrrheum.2012.1 (2012).
Yellowley, C. E. & Genetos, D. C. Hypoxia Signaling in the Skeleton: Implications for Bone Health. Curr Osteoporos Rep 17, 26-35, doi: 10.1007/s11914-019-00500-6 (2019).
Grundnes, O. & Reikerås, O. Blood flow and mechanical properties of healing bone. Femoral osteotomies studied in rats. Acta Orthop Scand 63, 487-491, doi: 10.3109/17453679209154720 (1992).
Schipani, E. et al. Hypoxia in cartilage: HIF-1alpha is essential for chondrocyte growth arrest and survival. Genes Dev 15, 2865-2876, doi: 10.1101/gad.934301 (2001).
Amarilio, R. et al. HIFla regulation of Sox9 is necessary to maintain differentiation of hypoxic prechondrogenic cells during early skeletogenesis. Development 134, 3917-3928, doi: 10.1242/dev.008441 (2007).
Kolar, P., Gaber, T., Perka, C., Duda, G. N. & Buttgereit, F. Human early fracture hematoma is characterized by inflammation and hypoxia. Clin Orthop Relat Res 469, 3118-3126, doi: 10.1007/s11999-011-1865-3 (2011).
Komatsu, D. E. & Hadjiargyrou, M. Activation of the transcription factor HIF-1 and its target genes, VEGF, HO-1, iNOS, during fracture repair. Bone 34, 680-688, doi: 10.1016/j.bone.2003.12.024 (2004).
Stegen, S. et al. Adequate hypoxia inducible factor 1α signaling is indispensable for bone regeneration. Bone 87, 176-186, doi: 10.1016/j.bone.2016.03.014 (2016).
Wan, C. et al. Activation of the hypoxia-inducible factor-1alpha pathway accelerates bone regeneration. Proc Natl Acad Sci USA 105, 686-691, doi: 10.1073/pnas.0708474105 (2008).
Lennon, D. P., Edmison, J. M. & Caplan, A. I. Cultivation of rat marrow-derived mesenchymal stem cells in reduced oxygen tension: effects on in vitro and in vivo osteochondrogenesis. J Cell Physiol 187, 345-355, doi: 10.1002/jcp. 1081 (2001).
Pattappa, G., Johnstone, B., Zellner, J., Docheva, D. & Angele, P. The Importance of Physioxia in Mesenchymal Stem Cell Chondrogenesis and the Mechanisms Controlling Its Response. Int J Mol Sci 20, doi: 10.3390/ijms20030484 (2019).
Aro, H., Eerola, E., Aho, A. J. & Niinikoski, J. Tissue oxygen tension in externally stabilized tibial fractures in rabbits during normal healing and infection. Journal of Surgical Research 37, 202-207, doi: 10.1016/0022-4804 (84) 90181-1 (1984).
Brighton, C. T. & Krebs, A. G. Oxygen tension of healing fractures in the rabbit. J Bone Joint Surg Am 54, 323-332 (1972).
Epari, D. R., Lienau, J., Schell, H., Witt, F. & Duda, G. N. Pressure, oxygen tension and temperature in the periosteal callus during bone healing—an in vivo study in sheep. Bone 43, 734-739, doi: 10.1016/j.bone.2008.06.007 (2008).
Lu, C. et al. Tibial fracture decreases oxygen levels at the site of injury. Iowa Orthop J 28, 14-21 (2008).
Lu, C. et al. The role of oxygen during fracture healing. Bone 52, 220-229, doi: 10.1016/j.bone.2012.09.037 (2013).
Boerckel, J. D., Uhrig, B. A., Willett, N. J., Huebsch, N. & Guldberg, R. E. Mechanical regulation of vascular growth and tissue regeneration in vivo. Proc Natl Acad Sci USA 108, E674-680, doi: 10.1073/pnas. 1107019108 (2011).
Stefanowski, J. et al. Spatial Distribution of Macrophages During Callus Formation and Maturation Reveals Close Crosstalk Between Macrophages and Newly Forming Vessels. Front Immunol 10, 2588, doi: 10.3389/fimmu.2019.02588 (2019).
Koch, C. J. Measurement of absolute oxygen levels in cells and tissues using oxygen sensors and 2-nitroimidazole EF5. Methods Enzymol 352, 3-31, doi: 10.1016/s0076-6879 (02) 52003-6 (2002).
Koch, C. J. Importance of antibody concentration in the assessment of cellular hypoxia by flow cytometry: EF5 and pimonidazole. Radiat Res 169, 677-688, doi: 10.1667/rr1305.1 (2008).
Lord, E. M., Harwell, L. & Koch, C. J. Detection of hypoxic cells by monoclonal antibody recognizing 2-nitroimidazole adducts. Cancer Res 53, 5721-5726 (1993).
Vanderkooi, J. M., Maniara, G., Green, T. J. & Wilson, D. F. An optical method for measurement of dioxygen concentration based upon quenching of phosphorescence. J Biol Chem 262, 5476-5482 (1987).
Lebedev, A. Y. et al. Dendritic phosphorescent probes for oxygen imaging in biological systems. ACS Appl Mater Interfaces 1, 1292-1304, doi: 10.1021/am9001698 (2009).
Esipova, T. V. et al. Two new “protected” oxyphors for biological oximetry: properties and application in tumor imaging. Anal Chem 83, 8756-8765, doi: 10.1021/ac2022234 (2011).
Holland, S. K., Kennan, R. P., Schaub, M. M., D'Angelo, M. J. & Gore, J. C. Imaging oxygen tension in liver and spleen by 19F NMR. Magnetic Resonance in Medicine 29, 446-458, doi.org/10.1002/mrm. 1910290405 (1993).
Stewart, I. B. & Mckenzie, D. C. The Human Spleen During Physiological Stress. Sports Medicine 32, 361-369, doi: 10.2165/00007256-200232060-00002 (2002).
Boerckel, J. D. Created in BioRender: https://BioRender.com/r21q019. (2025).
Kovtun, A. et al. The crucial role of neutrophil granulocytes in bone fracture healing. Eur Cell Mater 32, 152-162, doi: 10.22203/ecm.v032a10 (2016).

Schlundt, C. et al. Macrophages in bone fracture healing: Their essential role in endochondral ossification. Bone 106, 78-89, doi: 10.1016/j.bone.2015.10.019 (2018).
Pevny, L. et al. Erythroid differentiation in chimaeric mice blocked by a targeted mutation in the gene for transcription factor GATA-1. Nature 349, 257-260, doi: 10.1038/349257a0 (1991).
Yamamoto, M. et al. Activity and tissue-specific expression of the transcription factor NF-E1 multigene family. Genes Dev 4, 1650-1662, doi: 10.1101/gad.4.10.1650 (1990).
Chen, K. et al. Resolving the distinct stages in erythroid differentiation based on dynamic changes in membrane protein expression during erythropoiesis. Proceedings of the National Academy of Sciences 106, 17413-17418, doi: 10.1073/pnas.0909296106 (2009).
Yien, Y. Y. & Perfetto, M. Regulation of Heme Synthesis by Mitochondrial Homeostasis Proteins. Frontiers in Cell and Developmental Biology 10 (2022).
Zhang, F. et al. An extra-erythrocyte role of haemoglobin body in chondrocyte hypoxia adaption. Nature 622, 834-841, doi: 10.1038/s41586-023-06611-6 (2023).
Burmester, T., Weich, B., Reinhardt, S. & Hankeln, T. A vertebrate globin expressed in the brain. Nature 407, 520-523, doi: 10.1038/35035093 (2000).
Gödecke, A. et al. Disruption of myoglobin in mice induces multiple compensatory mechanisms. Proc Natl Acad Sci USA 96, 10495-10500, doi: 10.1073/pnas.96.18.10495 (1999).
Liu, L., Zeng, M. & Stamler, J. S. Hemoglobin induction in mouse macrophages. Proceedings of the National Academy of Sciences 96, 6643-6647, doi: 10.1073/pnas.96.12.6643 (1999).
Tusi, B. K. et al. Population snapshots predict early haematopoietic and erythroid hierarchies. Nature 555, 54-60, doi: 10.1038/nature25741 (2018).
Koulnis, M. et al. Identification and analysis of mouse erythroid progenitors using the CD71/TER119 flow-cytometric assay. J Vis Exp, doi: 10.3791/2809 (2011).
Hentze, M. W., Muckenthaler, M. U. & Andrews, N. C. Balancing Acts: Molecular Control of Mammalian Iron Metabolism. Cell 117, 285-297, doi.org/10.1016/S0092-8674 (04) 00343-5 (2004).
Ponka, P. Tissue-Specific Regulation of Iron Metabolism and Heme Synthesis: Distinct Control Mechanisms in Erythroid Cells. Blood 89, 1-25, doi: 10.1182/blood. V89.1.1 (1997).
Matos, M. A., Araújo, F. P. & Paixão, F. B. Histomorphometric evaluation of bone healing in rabbit fibular osteotomy model without fixation. Journal of Orthopaedic Surgery and Research 3, 4, doi: 10.1186/1749-799X-3-4 (2008).
Donneys, A. et al. Localized deferoxamine injection augments vascularity and improves bony union in pathologic fracture healing after radiotherapy. Bone 52, 318-325, doi: 10.1016/j.bone.2012.10.014 (2013).
Lang, A. et al. MIF does only marginally enhance the pro-regenerative capacities of DFO in a mouse-osteotomy-model of compromised bone healing conditions. Bone 154, 116247, doi: 10.1016/j.bone.2021.116247 (2022).
Chow, A. et al. CD169⁺ macrophages provide a niche promoting erythropoiesis under homeostasis and stress. Nat Med 19, 429-436, doi: 10.1038/nm.3057 (2013).
Ramos, P. et al. Macrophages support pathological erythropoiesis in polycythemia vera and β-thalassemia. Nat Med 19, 437-445, doi: 10.1038/nm.3126 (2013).
Batoon, L. et al. CD169 (+) macrophages are critical for osteoblast maintenance and promote intramembranous and endochondral ossification during bone repair. Biomaterials 196, 51-66, doi: 10.1016/j.biomaterials.2017.10.033 (2019).
McDermott, A. M. et al. Recapitulating bone development through engineered mesenchymal condensations and mechanical cues for tissue regeneration. Sci Transl Med 11, doi: 10.1126/scitranslmed.aav7756 (2019).
Gerber, H.-P. et al. VEGF couples hypertrophic cartilage remodeling, ossification and angiogenesis during endochondral bone formation. Nature Medicine 5, 623-628, doi: 10.1038/9467 (1999).
Maes, C. et al. Impaired angiogenesis and endochondral bone formation in mice lacking the vascular endothelial growth factor isoforms VEGF164 and VEGF188. Mech Dev 111, 61-73, doi: 10.1016/s0925-4773 (01) 00601-3 (2002).
Hankenson, K. D., Dishowitz, M., Gray, C. & Schenker, M. Angiogenesis in bone regeneration. Injury 42, 556-561, doi.org/10.1016/j.injury.2011.03.035 (2011).
Lang, A. et al. Cyr61 delivery promotes angiogenesis during bone fracture repair. bioRxiv, 2024.2004.2005.588239, doi: 10.1101/2024.04.05.588239 (2024).
van Gastel, N. et al. Lipid availability determines fate of skeletal progenitor cells via SOX9. Nature 579, 111-117, doi: 10.1038/s41586-020-2050-1 (2020).
Lambeth, J. D. NOX enzymes and the biology of reactive oxygen. Nature Reviews Immunology 4, 181-189, doi: 10.1038/nri1312 (2004).
Pedroza-Dávila, U. et al. Metabolism, ATP production and biofilm generation by Staphylococcus epidermidis in either respiratory or fermentative conditions. AMB Express 10, 31, doi: 10.1186/s13568-020-00966-z (2020).
Anup, A. et al. Embracing ethical research: Implementing the 3R principles into fracture healing research for sustainable scientific progress. J Orthop Res 42, 568-577, doi: 10.1002/jor.25741 (2024).
Röntgen, V. et al. Fracture healing in mice under controlled rigid and flexible conditions using an adjustable external fixator. J Orthop Res 28, 1456-1462, doi: 10.1002/jor.21148 (2010).
Jirkof, P. et al. Administration of Tramadol or Buprenorphine via the drinking water for post-operative analgesia in a mouse-osteotomy model. Sci Rep 9, 10749, doi: 10.1038/s41598-019-47186-5 (2019).
Wolter, A. et al. A buprenorphine depot formulation provides effective sustained post-surgical analgesia for 72 h in mouse femoral fracture models. Sci Rep 13, 3824, doi: 10.1038/s41598-023-30641-9 (2023).
Chong, S. H. et al. Real-time tracking of brain oxygen gradients and blood flow during functional activation. Neurophotonics 9, 045006, doi: 10.1117/1.NPh.9.4.045006 (2022).
Friedman, E. S. et al. Microbes vs. chemistry in the origin of the anaerobic gut lumen. Proc Natl Acad Sci USA 115, 4170-4175, doi: 10.1073/pnas. 1718635115 (2018).
El Khatib, M. et al. Ultrafast Tracking of Oxygen Dynamics During Proton FLASH. Int J Radiat Oncol Biol Phys 113, 624-634, doi: 10.1016/j.ijrobp.2022.03.016 (2022).
Hao, Y. et al. Integrated analysis of multimodal single-cell data. Cell 184, 3573-3587.e3529, doi: 10.1016/j.cell.2021.04.048 (2021).

What is claimed:

1. A device for bone fracture or segmental defect healing, comprising:
an implantable substrate;
a first active composition, the first active composition optionally in contact with the implantable substrate; and
a second active composition, the second active composition optionally in contact with the implantable substrate,
wherein the first active composition comprises one or more compositions for bone healing, and
wherein the second active composition comprises one or more oxygen-modulating compositions.

2. The device of claim 1, wherein the implantable substrate is any one or more of an orthopedic implant, a scaffold, a gel, a putty, a gel and putty, granules, a sponge, a foam, demineralized bone matrix particles, fibers, flexible strips, a graft, an injectable bone graft substitute, a collection of microparticles, or a collection of nanoparticles.

3. The device of claim 1, wherein the implantable substrate comprises a biocompatible material.

4. The device of claim 1, wherein the implantable substrate comprises a biodegradable or non-biodegradable material.

5. The device of claim 1, wherein the implantable substrate is a scaffold.

6. The device of claim 1, wherein the implantable substrate comprises a material selected from the group consisting of polylactic acid, polyglycolic acid, PLGA polymers, alginates and alginate derivatives, gelatin, collagen, fibrin, hyaluronic acid, hydroxyapatite, calcium phosphate, tri-calcium phosphate, demineralized bone matrix, calcium sulfate, collagen matrix, laminin-rich gels, agarose, natural and synthetic polysaccharides, polyamino acids, polypeptides, polyesters, polyanhydrides, polyphosphazines, poly(vinyl alcohols), poly(alkylene oxides), poly(allylamines) (PAM), poly(acrylates), modified styrene polymers, pluronic polyols, polyoxamers, poly(uronic acids), poly(vinylpyrrolidone), and copolymers or graft copolymers thereof.

7. The device of claim 1, wherein the implantable substrate is coated or seeded with at least one of the first active composition and the second active composition.

8. The device of claim 1, wherein the first active composition comprises any one or more of a cell, an extracellular matrix protein, a growth factor, a cytokine, or a chemoattractant.

9. The device of claim 1, wherein the first active composition comprises any one or more of erythropoietin (Epo), stem cell factor (SCF), bone morphogenetic protein 4 (BMP-4), or growth/differentiation factor 15 (GDF-15).

10. The device of claim 1, wherein the second active composition comprises any one or more of antibodies, hypoxia inducing factor (HIF) stabilizers, transferrin blocking agents, iron metabolism modulators, hemoglobin inhibitors, or iron chelators.

11. The device of claim 1, wherein the second active composition comprises any one or more of anti-cluster differentiation-71 (anti-CD-71) antibody, anti-CD-71 fragment antigen-binding (Fab), anti-CD-71 single chain variable fragment (scFv), anti-CD-71 diabody, or an iron chelator.

12. A method of enhancing healing at a bone fracture site in a subject, comprising: administering at the bone fracture site a device according to claim 1.

13. The method of claim 12, comprising administering the device at the bone fracture site at a time of surgery.

14. The method of claim 12, wherein the bone fracture comprises any one or more of a fracture or segmental defect of long bone, a fracture or segmental defect of a rib, a fracture or fusion of vertebrae, an osteoporotic fracture, or a mandibular defect.

15. The method of claim 12, wherein the implantable substrate is seeded with autologous or allogeneic bone marrow cells.

16. The method of claim 12, wherein an oxygenation level at the bone fracture or segmental defect site is reduced after administering the device.

17. The method of claim 12, wherein the enhancing of bone fracture or segmental defect healing comprises at least one of decreasing tissue oxygen tension or increasing a number of hypoxic cells at the bone fracture or segmental defect site.

18. The method of claim 12, wherein the enhancing of bone fracture or segmental defect healing comprises an increase in osteoprogenitor activation and angiogenesis at the bone fracture or segmental defect site; an increase in bone volume at the bone fracture or segmental defect site; and/or achieving union in at least 90% of bone fracture or segmental defect sites with the administered device.

19. The method of claim 12, wherein the enhancing of bone fracture or segmental defect healing comprises reducing time to union at the bone fracture or segmental defect site.

20. A method of reducing incidence of non-union among subjects with bone fracture, comprising:
administering the device of claim 1 at the bone fracture site of each subject, wherein the incidence of non-union at the bone fracture site is reduced to below 5% among the subjects.
